(19) United States
(12) Patent Application Publication
(10) Pub. No.: US 2024/0335523 A1
(43) Pub. Date: Oct. 10, 2024

(54) Glycoengineered Foldon Domains and Related Compositions and Methods

(71) Applicant: The Regents of the University of California, Oakland, CA (US)

(72) Inventors: Rebecca Dubois, Santa Cruz, CA (US); John Dzimianski, Santa Cruz, CA (US)

(21) Appl. No.: 18/294,912

(22) PCT Filed: Aug. 4, 2022
(86) PCT No.: PCT/US2022/074549

§ 371 Date: Feb. 2, 2024

Related U.S. Application Data
(60) Provisional application No. 63/229,912, filed on Aug. 5, 2021.

(51) Int. Cl. A61K 39/145; A61K 39/12; A61K 39/21; A61K 39/215; C07K 14/005

(52) U.S. Cl. CPC A61K 39/145; A61K 39/12; A61K 39/21; A61K 39/215; C07K 14/005; C07K 2319/20; C07K 2319/91; C12N 2760/16122; C12N 2760/16134

(57) ABSTRACT

Provided are polypeptides comprising, consisting essentially of, or consisting of a foldon domain comprising an N-linked glycosylation motif. In some embodiments, the foldon domain is fused to a heterologous amino acid sequence, non-limiting examples of which include immunogenic polypeptides. Trimers of three polypeptides of the present disclosure are also provided, as are related immunogenic compositions and methods of inducing an immune response in an individual. Also included are vectors, e.g., cloning and/or expression vectors that include a nucleic acid sequence encoding a foldon as disclosed herein.

### CROSS-REFERENCE TO RELATED APPLICATIONS

This application claims priority benefit to U.S. provisional application Ser. No. 63/229,912 filed on Aug. 5, 2021, which application is incorporated herein by reference in its entirety.

### STATEMENT OF GOVERNMENT SUPPORT

This invention was made with Government support under contract 75N93019C00052 awarded by the National Institutes of Health. The Government has certain rights in the invention.

### INTRODUCTION

Vaccine design remains at the forefront of therapeutic development. Reducing, and potentially eradicating, severe infectious diseases relies on vaccines that elicit effective, long-lasting immunity. A major hurdle to achieving these goals in virology is variable glycosylation patterns on virus proteins. Glycosylation shields important epitopes from antibody recognition. As a result of antigenic drift, viruses such as HIV and Influenza undergo alterations in the glycosylation of their surface proteins, allowing future virus strains to evade immunity induced by previous infection. Recently, however, new strategies have been uncovering ways to exploit this same mechanism against viruses through targeted glycoengineering.

By modifying the glycosylation patterns of virus proteins, antibody responses can be specifically biased to recognize particular epitopes. Investigations with the HIV Env protein have demonstrated that glycoengineering to obscure particular antigenic sites can be used to elicit more broadly reactive neutralizing antibodies or divert the immune response towards previously under-recognized epitopes. In Influenza, immune shielding by glycosylation has been used to bias immune responses towards the more highly conserved stem domain. In a study on Zika virus, glycan-shielding a non-neutralizing epitope resulted in a more robust neutralizing antibody response.

### SUMMARY

Provided are polypeptides comprising, consisting essentially of, or consisting of a foldon domain comprising an N-linked glycosylation motif. In some embodiments, the foldon domain is fused to a heterologous amino acid sequence, non-limiting examples of which include immunogenic polypeptides. Trimers of three polypeptides of the present disclosure are also provided, as are related immunogenic compositions and methods of inducing an immune response in an individual.

The polypeptides and related compositions and methods of the present disclosure find use in a variety of applications. For example, with the benefit of the present disclosure, it is expected that glycan-shielding of foldon domains would result in improved recombinant vaccines. Potential recombinant vaccine candidates, such as the influenza hemagglutinin (HA, e.g., HA1 and/or HA2), HIV Env, and RSV F proteins, are expressed as soluble proteins lacking the native transmembrane domain. These proteins are often fused with trimerization domains, such as the GCN4 isoleucine zipper (IZ) or the T4 bacteriophage fibritin foldon (“foldon” or “FOLDON”) domains, to stabilize the native oligomeric state. However, these domains themselves elicit antibody responses in animal models, raising concerns regarding the risks of unintended immunogenicity. With foldon-containing Computationally Optimized Broadly Reactive Antigen (COBRA) influenza HA proteins (as one example) showing great promise as recombinant vaccine candidates, there is a need to eliminate potential off-target effects. The polypeptides and related compositions and methods of the present disclosure address this and other needs by reducing foldon immunogenicity through structure-based glycoengineering.

### BRIEF DESCRIPTION OF THE FIGURES

FIG. 1 depicts structural validation and glycoengineering of COBRA HA antigens.

FIG. 2 illustrates structural analysis of COBRA HA antigens.

FIG. 3 provides a schematic of a fusion protein comprising HA1 and HA2, foldon domain, AviTag, and a 6×His tag.

FIG. 4 provides a schematic for foldon glycoengineering.

### DEFINITIONS

All publications, patents and patent applications cited herein are hereby incorporated by reference in their entireties.

It must be noted that, as used in this specification and the appended claims, the singular forms “a”, “an” and “the” include plural referents unless the content clearly dictates otherwise. Thus, for example, reference to “an N-glycosylation motif” includes one or more such motifs, and the like. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

As used herein, the term “cell line” refers to a population of cells produced from a single cell and therefore consisting of cells with a uniform genetic makeup.

A “conservative substitution” is one in which an amino acid is substituted for another amino acid that has similar properties, such that one skilled in the art of peptide chemistry would expect the secondary structure and hydropathic nature of the cell surface receptor, protease, or domain thereof, to be substantially unchanged. Modifications may be made in the structure of the polynucleotides and polypeptides contemplated in particular embodiments. Polypeptides including one or more conservative substitutions still obtain a functional molecule (e.g., capable of trimerization) that encodes a variant or derivative polypeptide with desirable characteristics, e.g., reduced immunogenicity. When it is desired to alter the amino acid sequence of a foldon domain (e.g., a wild-type foldon domain) to create an equivalent or improved variant foldon domain, one skilled in the art, for example, can change one or more of the codons of the encoding DNA sequence.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. “Heterologous” in the context of recombinant cells can refer to the presence of a nucleic acid (or gene product, such as a polypeptide) that is of a different genetic origin than the host cell in which it is present. For example, a recombinant cell expressing a heterologous polypeptide refers to a cell that is genetically modified to introduce a nucleic acid encoding the polypeptide which nucleic acid is not naturally present in the cell.

A “host cell,” as used herein, denotes an in vitro prokaryotic cell (e.g., bacterial cell, such as, E. coli) or an eukaryotic cell (e.g., a mammalian cell, such as, a CHO cell line), which cell can be, or has been, used as a recipient for a nucleic acid, and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject eukaryotic host cell is a genetically modified eukaryotic host cell, by virtue of introduction into of a heterologous nucleic acid, e.g., an exogenous nucleic acid that is foreign to the eukaryotic host cell, or a recombinant nucleic acid that is not normally found in the eukaryotic host cell.

The term “operably linked” refers to a juxtaposition wherein the components so described are in a relationship permitting them to function in their intended manner. For instance, a promoter is operably linked to a nucleotide sequence if the promoter affects the transcription or expression of the nucleotide sequence.

The terms “polynucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term also includes modifications, such as by methylation and/or by capping, and unmodified forms of the polynucleotide. More particularly, the terms “polynucleotide,” “nucleic acid” and “nucleic acid molecule” include polydeoxyribonucleotides (containing 2 deoxy D ribose), polyribonucleotides (containing D ribose), any other type of polynucleotide which is an N- or C glycoside of a purine or pyrimidine base, and other polymers containing nonnucleotidic backbones, for example, polyamide (e.g., peptide nucleic acids (PNAs)) and polymorpholino (commercially available from the Anti Virals, Inc., Corvallis, Oregon, as Neugene) polymers, and other synthetic sequence specific nucleic acid polymers providing that the polymers contain nucleobases in a configuration which allows for base pairing and base stacking, such as is found in DNA and RNA. There is no intended distinction in length between the terms “polynucleotide,” “nucleic acid” and “nucleic acid molecule,” and these terms will be used interchangeably.

The terms “polypeptide”, “peptide”, or “protein” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof.

### DETAILED DESCRIPTION

### Mutant Foldon Domains and Polypeptides

Aspects of the present disclosure include foldon domains that have been engineered to include one or more N-linked glycosylation motifs. A foldon domain that is glycosylated may produce a lower immune response, when administered to a subject, as compared to a foldon domain that is not glycosylated. Foldon domains may be used for presentation of polypeptides against which generation of an immune response is desired.

Aspects of the present disclosure further include polypeptides comprising, consisting essentially of, or consisting of a foldon domain comprising an N-linked glycosylation motif. In certain embodiments, the foldon domain is used for presenting a polypeptide in a trimeric form. Accordingly, a trimer comprises three polypeptides, where each polypeptide is a fusion of an antigen, against which an immune response is desired, and a foldon domain is provided, wherein the foldon domain is a mutant foldon domain comprising at least one N-linked glycosylation motif.

In certain embodiments, a foldon domain comprising an N-linked glycosylation motif comprises an amino acid sequence having 85% or greater identity (e.g., 90% identity) to the amino acid sequence set forth in SEQ ID NO:1 (GYIPEAPRDGQAYVRKDGEWVLLSTFL), and wherein the N-linked glycosylation motif is NXS/T, where X is any amino acid other than proline.

According to some embodiments, a foldon domain comprising an N-linked glycosylation motif comprises an amino acid sequence at least 85% identical to the amino acid sequence of SEQ ID NO:1 and comprises at least one of the following pairs of substitutions of the corresponding amino acids in SEQ ID NO: 1:
R8N and G10T/S;
G10N and A12T/S;
K16N and G18T/S;
D17N and E19T/S;
E19N and V21T/S; and
S24N and F26T.

In certain embodiments, a foldon domain comprising an N-linked glycosylation motif comprises an amino acid sequence at least 85% identical to the amino acid sequence of SEQ ID NO:1 and comprises at least two of the following pairs of substitutions of the corresponding amino acids in SEQ ID NO: 1:
R8N and G10T/S;
K16N and G18T/S; and
S24N and F26T;
or
G10N and A12T/S;
K16N and G18T/S;
E19N and V21T/S; and
S24N and F26T;
or
R8N and G10T/S;
G10N and A12T/S;
E19N and V21T/S; and
S24N and F26T.

According to some embodiments, the N-linked glycosylation motif is NDT/S, NGT/S, NQT/S, NTT/S, or NWT/S.

In certain embodiments, the foldon domain comprises an amino acid sequence having 85% or greater identity (e.g., 90% identity) to the amino acid sequence set forth in:

SEQ ID NO: 2
(GYIPEAPNDTQAYVRKDGEWVLLSTFL);
SEQ ID NO: 3
(GYIPEAPRDNQSYVRKDGEWVLLSTFL);
SEQ ID NO: 4
(GYIPEAPRDGQAYVRKDGNWTLLSTFL);
SEQ ID NO: 5
(GYIPEAPRDGQAYVRNDTEWVLLSTFL);
SEQ ID NO: 6
(GYIPEAPRDGQAYVRKNGTWVLLSTFL);
or
SEQ ID NO: 7
(GYIPEAPRDGQAYVRKDGEWVLLNTTL).

According to some embodiments, the foldon domain comprises an amino acid sequence having the amino acid sequence set forth in any one of SEQ ID NOs: 2 to 7 or comprises the amino acid sequence set forth in any one of SEQ ID NOs: 2 to 7 with one, two, three, or four conservative amino acid substitutions, where the foldon domain mediates formation of a timer of a polypeptide comprising the foldon domain.

In certain embodiments, a polypeptide of the present disclosure comprising a foldon domain comprising at least one N-linked glycosylation motif as provided herein comprises an amino acid sequence that is heterologous to the foldon domain. In certain embodiments, the polypeptide may be a fusion of the heterologous amino acid sequence and the foldon domain, where the C-terminus of the heterologous amino acid sequence is fused to the N-terminus of the foldon domain. In certain embodiments, the heterologous amino acid sequence may be an immunogenic polypeptide. In certain embodiments, the immunogenic polypeptide may be any polypeptide against which an immune response is desired. In certain embodiments, the immunogenic polypeptide may be a polypeptide that is present on surface of a cell or a viral particle. In certain embodiments, the immunogenic polypeptide may be a polypeptide that is present on surface of a cell as a trimeric polypeptide. In certain embodiments, the immunogenic polypeptide may be a polypeptide that is present on surface of a cell as a trimeric polypeptide and is engineered to be a soluble polypeptide that lacks a transmembrane domain present in the wild-type polypeptide. In certain embodiments, the immunogenic polypeptide may be a vaccine candidate. In certain embodiments, the immunogenic polypeptide may be a viral polypeptide. In certain embodiments, the viral immunogenic polypeptide may include an influenza hemagglutinin (HA) polypeptide, a human immunodeficiency virus (HIV) Env polypeptide, a respiratory syncytial virus (RSV) F polypeptide, or a SARS-CoV-2 spike polypeptide. In certain embodiments, the influenza HA polypeptide comprises an HA1 domain, an HA2 domain, or both. In certain embodiments, the influenza HA polypeptide comprises a Computationally Optimized Broadly Reactive Antigen (COBRA) influenza HA polypeptide. In certain embodiments, the HIV Env polypeptide comprises gp120, e.g., gp120 derived from a Clade B strain of HIV. In certain embodiments, the HIV Env polypeptide comprises gp160, gp120 (e.g., gp120 monomer), gp140 (e.g., gp140 trimer) or an envelope gp fragment containing variable regions 1 and 2 (V1/V2).

According to some embodiments, the polypeptide may include a tag, e.g., a purification tag. In certain embodiments, the polypeptide may include a signal peptide to facilitate secretion of the polypeptide. In certain embodiments, the polypeptide when expressed in a host cell forms a trimer. In certain embodiments, the signal peptide may be herpes simplex virus glycoprotein D (gD) or a tissue plasminogen activator (tPA) signal sequence.

In certain embodiments, the polypeptide is glycosylated. In certain embodiments, the glycosylation comprises N-acetylglucosamine (GlcNAc) in the β-configuration.

According to some embodiments, a trimer of three polypeptides as disclosed herein is provided. The sequences of each of the polypeptide in the timer may be identical or may differ in the foldon domain. In certain embodiments, one or more of the N-linked glycosylation motifs of the foldon domains are glycosylated.

### Immunogenic Compositions

Also provided by the present disclosure are immunogenic compositions comprising the trimers disclosed herein and a pharmaceutically acceptable excipient. The pharmaceutically acceptable excipient may comprise an adjuvant. Examples of known suitable adjuvants that can be used in humans include, but are not necessarily limited to, an aluminum adjuvant (e.g., aluminum phosphate, or aluminum hydroxide), MF59 (4.3% w/v squalene, 0.5% w/v Tween 80™, 0.5% w/v Span 85), a CpG-containing nucleic acid (where the cytosine is unmethylated), QS21, MPL, 3DMPL, extracts from Aquilla, ISCOMS, LT/CT mutants, poly(D,L-lactide-co-glycolide) (PLG) microparticles, Quil A, interleukins, and the like. For experimental animals, one can use Freund's adjuvant (incomplete Freund's adjuvant; complete Freund's adjuvant), N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogenic antigen or antigenic epitope thereof.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (Detox™); (2) saponin adjuvants, such as QS21 or Stimulon™ (Cambridge Bioscience, Worcester, Mass.) may be used or particles generated therefrom such as ISCOMs (immunostimulating complexes), which ISCOMS may be devoid of additional detergent e.g. WO 00/07621; (3) Complete Freund's Adjuvant (CFA) or Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO 00/56358; (6) combinations of 3dMPL with, for example, QS21 and/or oil-in-water emulsions e.g. EP-A-0835318, EP-A-0735898, EP-A-0761231; (7) oligonucleotides comprising CpG motifs (see, e.g., WO 98/52581), e.g., an oligonucleotide containing at least one CG dinucleotide, where the cytosine is unmethylated; (8) a polyoxyethylene ether or a polyoxyethylene ester (see, e.g. WO 99/52549); (9) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (10) a saponin and an immunostimulatory oligonucleotide (e.g. a CpG oligonucleotide) (WO 00/62800); (11) an immunostimulant and a particle of metal salt e.g. WO 00/23105; (12) a saponin and an oil-in-water emulsion e.g. WO 99/11241; (13) a saponin (e.g. QS21)+3dMPL+1M2 (optionally+a sterol) e.g. WO 98/57659; (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc. Adjuvants suitable for administration to a human are of particular interest. In some cases, the adjuvant is an aluminum salt adjuvant (e.g., aluminum phosphate or aluminum hydroxide).

The immunogenic compositions may contain other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of the timer in a formulation can vary widely (e.g., from less than about 0.1%, e.g., at or at least about 2% to as much as 20% to 50% or more by weight) and will usually be selected primarily based on fluid volumes, viscosities, and patient-based factors in accordance with the particular mode of administration selected and the patient's immune response.

The trimer-containing formulations may be provided in the form of a solution, suspension, powder, gel, aerosol or the like.

The trimer-containing formulations may also be provided so as to enhance serum half-life of the trimer following administration. For example, where isolated trimers are formulated for injection, the trimer may be provided in a liposome formulation, prepared as a colloid, or other conventional techniques for extending serum half-life. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The preparations may also be provided in controlled release or slow-release forms.

### Nucleic Acids and Vectors

Aspects of the present disclosure include a nucleic acid comprising a sequence encoding a foldon domain comprising at least one (e.g., two or three) N-linked glycosylation motifs. The nucleic acid may be present in a vector, e.g., a cloning vector or an expression vector.

A cloning vector may be used to introduce a sequence encoding an antigen of interest upstream of the sequence encoding the foldon domain. An expression vector may also be used for introducing a sequence encoding an antigen of interest upstream of the sequence encoding the foldon domain.

Aspects of the present disclosure include a nucleic acid comprising a sequence encoding an antigen fused in frame to a sequence encoding a foldon domain comprising at least one (e.g., two or three) N-linked glycosylation motifs.

### Methods of Inducing an Immune Response

The present disclosure provides a method of inducing an immune response to the heterologous amino acid sequence, e.g., an antigen fused to foldon domain in the trimer in a mammalian subject. The methods generally involve administering to an individual in need thereof an effective amount of a subject immunogenic composition.

The trimer-containing antigenic compositions are generally administered to a human subject that is at risk of acquiring a disease so as to prevent or at least partially arrest the development of disease and its complications. An amount adequate to accomplish this is defined as a “therapeutically effective dose.” Amounts effective for therapeutic use will depend on, e.g., the antigenic composition, the manner of administration, the weight and general state of health of the patient, and the judgment of the prescribing physician. Single or multiple doses of the antigenic compositions may be administered depending on the dosage and frequency required and tolerated by the patient, and route of administration.

The trimer-containing antigenic compositions are generally administered in an amount effective to elicit an immune response, particularly a humoral immune response, e.g., an antibody response, in the host. As noted above, amounts for immunization will vary, and can generally range from about 1 μg to 100 μg per 70 kg patient, usually 5 μg to 50 μg/70 kg. Substantially higher dosages (e.g. 10 mg to 100 mg or more) may be suitable in oral, nasal, or topical administration routes. The initial administration can be followed by booster immunization. Vaccination in some cases involves at least one booster, and in some cases two boosters.

In general immunization can be accomplished by administration by any suitable route, including administration of the composition orally, nasally, nasopharyngeally, parenterally, enterically, gastrically, topically, transdermally, subcutaneously, intramuscularly, in tablet, solid, powdered, liquid, aerosol form, locally or systemically, with or without added excipients. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

An anti-antigen immune response can be assessed by known methods (e.g., by obtaining serum from the individual before and after the initial immunization, and demonstrating a change in the individual's immune status, for example an immunoprecipitation assay, an ELISA, or a bactericidal assay, a Western blot assay, or flow cytometric assay, or the like).

### Methods of Producing Trimers

The present disclosure provides a method of producing a trimer comprising the polypeptide described herein. The method may include culturing a recombinant host cell comprising an expression vector encoding the polypeptide under conditions suitable for expression of polypeptides comprising the foldon domains, glycosylation of the N-linked glycosylation motifs of the foldon domains, and trimerization of the polypeptides.

The method may also include separating the host cell line from the culture medium, to generate a cell culture comprising a secreted timer. Separating the host cells from the culture medium can be accomplished by methods known in the art, such as centrifugation, filtration, and the like. The trimer may be purified by any suitable method.

The host cell may be a mammalian cell that is capable of glycosylating a polypeptide comprising an N-linked glycosylation motif. In certain embodiments, the host cell may be a HEK 293 cell, a 293T cell, a CHO cell, and the like.

Notwithstanding the appended claims, the present disclosure is also defined by the following embodiments:

1. A polypeptide comprising a foldon domain comprising an N-linked glycosylation motif.

2. The polypeptide of embodiment 1, wherein the amino acid sequence of the foldon domain comprises 85% or greater identity to the amino acid sequence set forth in SEQ ID NO:1 (GYIPEAPRDGQAYVRKDGEWVLLSTFL), and
wherein the N-linked glycosylation motif is NXS/T, where X is any amino acid other than proline, and
optionally the N-linked glycosylation motif is NDT/S, NGT/S, NQT/S, NTT/S, or NWT/S.

3. The polypeptide of embodiment 2, wherein the N-linked glycosylation motif is NDT or NDS.

4. The polypeptide of embodiment 3, wherein the foldon domain comprises the amino acid sequence set forth in SEQ ID NO:2 (GYIPEAPNDTQAYVRKDGEWVLLSTFL).

5. The polypeptide of embodiment 2, wherein the N-linked glycosylation motif is NQS or NQT.

6. The polypeptide of embodiment 5, wherein the foldon domain comprises the amino acid sequence set forth in SEQ ID NO:3 (GYIPEAPRDNQSYVRKDGEWVLLSTFL).

7. The polypeptide of embodiment 2, wherein the N-linked glycosylation motif is NWT or NWS.

8. The polypeptide of embodiment 7, wherein the foldon domain comprises the amino acid sequence set forth in SEQ ID NO:4 (GYIPEAPRDGQAYVRKDGNWTLLSTFL).

9. The polypeptide of embodiment 1, wherein the foldon domain comprises the amino acid sequence set forth in:

SEQ ID NO: 5:
GYIPEAPRDGQAYVRNDTEWVLLSTFL;
SEQ ID NO: 6:
GYIPEAPRDGQAYVRKNGTWVLLSTFL;
or
SEQ ID NO: 7:
GYIPEAPRDGQAYVRKDGEWVLLNTTL.

10. The polypeptide of any one of embodiments 1 to 9, wherein the polypeptide consists essentially of the foldon domain.

11. The polypeptide of any one of embodiments 1 to 9, wherein the polypeptide consists of the foldon domain.

12. The polypeptide of any one of embodiments 1 to 9, wherein the foldon domain is fused to a heterologous amino acid sequence.

13. The polypeptide of embodiment 12, wherein the heterologous amino acid sequence is N-terminal to the foldon domain.

14. The polypeptide of embodiment 12 or embodiment 13, wherein the heterologous amino acid sequence is of an immunogenic polypeptide.

15. The polypeptide of embodiment 14, wherein the immunogenic polypeptide comprises a viral immunogenic polypeptide.

16. The polypeptide of embodiment 15, wherein the viral immunogenic polypeptide comprises an influenza hemagglutinin (HA) polypeptide, a human immunodeficiency virus (HIV) Env polypeptide, a respiratory syncytial virus (RSV) F polypeptide, or a SARS-CoV-2 spike polypeptide.

17. The polypeptide of embodiment 15, wherein the viral immunogenic polypeptide comprises an influenza HA polypeptide.

18. The polypeptide of embodiment 17, wherein the influenza HA polypeptide comprises an HA1 domain, an HA2 domain, or both.

19. The polypeptide of embodiment 17 or embodiment 18, wherein the influenza HA polypeptide comprises a Computationally Optimized Broadly Reactive Antigen (COBRA) influenza HA polypeptide.

20. The polypeptide of any one of embodiments 1 to 19, wherein the polypeptide comprises an affinity tag.

21. The polypeptide of any one of embodiments 1 to 20, wherein the polypeptide comprises a biotin tag.

22. The polypeptide of any one of embodiments 1 to 21, wherein the N-linked glycosylation motif is glycosylated.

23. The polypeptide of any one of embodiments 1 to 22, wherein the foldon domain comprises a plurality of N-linked glycosylation motifs.

24. The polypeptide of any one of embodiments 1 to 23, wherein the foldon domain comprises at least two N-linked glycosylation motifs selected from the group consisting of: NDT/S, NGT/S, NQT/S, NTT/S, and NWT/S.

25. The polypeptide of any one of embodiments 1 to 23, wherein the foldon domain comprises an amino acid sequence at least 85% identical to the amino acid sequence of SEQ ID NO:1 and comprises at least one of the following pairs of substitutions of the corresponding amino acids in SEQ ID NO: 1:
R8N and G10T/S;
G10N and A12T/S;
K16N and G18T/S;
D17N and E19T/S;
E19N and V21T/S; and
S24N and F26T.

26. The polypeptide of any one of embodiments 1 to 25, wherein the foldon domain comprises at least two of the following pairs of substitutions of the corresponding amino acids in SEQ ID NO: 1:
R8N and G10T/S;
K16N and G18T/S; and
S24N and F26T;
or
G10N and A12T/S;
K16N and G18T/S;
E19N and V21T/S; and
S24N and F26T;
or
R8N and G10T/S;
G10N and A12T/S;
E19N and V21T/S; and
S24N and F26T.

27. A trimer of three polypeptides of any one of embodiments 1 to 26 trimerized via their foldon domains.

28. A trimer of embodiment 27, wherein the foldon domains have different sequences.

29. The trimer of embodiment 27 or 28, wherein one or more of the N-linked glycosylation motifs of the foldon domains are glycosylated.

30. An immunogenic composition comprising the trimer of any one of embodiments 27-29 and a pharmaceutically acceptable excipient.

31. A method of inducing an immune response in an individual, comprising administering to the individual an effective amount of the immunogenic composition of embodiment 30.

32. A nucleic acid encoding the polypeptide of any one of embodiments 1 to 26.

33. An expression vector comprising the nucleic acid of embodiment 32 operably linked to a promoter.

34. A recombinant host cell comprising the nucleic acid of embodiment 32 or the expression vector of embodiment 33.

35. A method of producing trimers comprising glycosylated foldon domains, the method comprising:
culturing a recombinant host cell comprising the expression vector of embodiment 33 under conditions suitable for expression of polypeptides comprising the foldon domains, glycosylation of the N-linked glycosylation motifs of the foldon domains, and trimerization of the polypeptides.

36. The method according to embodiment 35, further comprising purifying the trimers.

37. A kit comprising the immunogenic composition of embodiment 30, optionally wherein the composition is present in one or more (e.g., two or more) unit dosages.

38. The kit of embodiment 37, further comprising instructions for administering to an individual an effective amount of the immunogenic composition to induce an immune response in the individual.

39. A foldon domain comprising or consisting of an amino acid sequence having at least 85% identity to SEQ ID NO: 1 and comprising at least one of the following pairs of substitutions of the corresponding amino acids in SEQ ID NO: 1:
R8N and G10T/S;
G10N and A12T/S;
K16N and G18T/S;
D17N and E19T/S;
E19N and V21T/S; and
S24N and F26T.

40. The foldon domain of embodiment 39, wherein the foldon domain comprises at least two of the following pairs of substitutions of the corresponding amino acids in SEQ ID NO: 1:
R8N and G10T/S;
K16N and G18T/S; and
S24N and F26T;
or
G10N and A12T/S;
K16N and G18T/S;
E19N and V21T/S; and
S24N and F26T;
or
R8N and G10T/S;
G10N and A12T/S;
E19N and V21T/S; and
S24N and F26T.

41. The foldon domain of embodiment 39 or 40, wherein the foldon domain comprises or consists of the amino acid sequence set forth in SEQ ID NO: 2, 3, 4, 5, 6 or 7.

42. A nucleic acid comprising a nucleotide sequence encoding the foldon domain of any one of embodiments 39-41 or as set forth in any one of embodiments 1-26.

43. A vector comprising the nucleic acid sequence of embodiment 42.

44. The vector of embodiment 43, comprising one or more restriction sites upstream of the nucleotide sequence, wherein the one or more restriction sites are configured for in-frame fusion of a nucleic acid encoding an immunogenic polypeptide.

45. A host cell comprising the vector of embodiments 43 or 44.

46. The host cell of embodiment 45, wherein the vector is a cloning vector and the host cell is a bacterial cell.

47. The host cell of embodiment 45, wherein the vector is an expression vector and the host cells is a mammalian cell.

48. The host cell of embodiment 47, wherein the mammalian cell is a CHO cell line.

49. A foldon domain of SEQ ID NO: 1 or an analog thereof, said analog comprising no more than 3 conservative amino acid substitutions comprising an engineered asparagine residue.

50. The foldon domain of embodiment 49 comprising an R8→N mutation; a G10→N mutation or an E18→N mutation.

51. The foldon domain of embodiment 50 comprising an R8→N mutation further comprising a G10→T mutation.

52. The foldon domain of embodiment 49 comprising a G10→N mutation further comprising a S12→T mutation.

53. The foldon domain of embodiment 49 comprising an E18→N mutation further comprising a V20→T mutation.

54. The foldon domain of embodiments 49-53 comprising a sequence selected from SEQ ID NO: 2, SEQ ID NO: 3, or SEQ ID NO: 4.

55. A protein construct comprising the foldon domain of any of embodiments 49-54 and a viral antigen.

56. The protein construct of embodiment 55 wherein one or more of the asparagine residues of the foldon domain of any of embodiments 49-54 is glycosylated.

57. The protein construct of embodiment 55 or embodiment 56 where the viral antigen comprises influenza HA.

58. A vaccine composition comprising the protein construct of any one of embodiments 55-57.

59. A nucleic acid that encodes the foldon domain of any one of embodiments 49-54.

60. A nucleic acid that encodes the protein construct of embodiments 55-57.

61. A plasmid comprising the nucleic acids of embodiments 59-60.

62. A mammalian cell line comprising the plasmid of embodiment 61.

63. The mammalian cell line of embodiment 62 where the cell line is a CHO cell line.

EXAMPLES

### Example 1: Mutant Foldon Domains

Using the known structure of the foldon domain, solvent exposed sites for mutagenesis were used to engineer an N-linked glycosylation motif into the foldon domain. Plasmids containing nucleic acid sequences encoding mutant foldon domains (SEQ ID NO: 2, SEQ ID NO: 3, and SEQ ID NO: 4) were cloned, transiently transfected into CHO cells, and the mutant foldon domains expressed. Wild type and mutant foldon domains were purified and mice were immunized with each. Mouse plasma will be analyzed for anti-foldon antibodies using enzyme-linked immunosorbent assay (ELISA) or biolayer interferometry-immunosorbent assay (BLI-SA).

Based on a crystal structure containing the foldon domain (PDB entry 1AVY), the trimeric foldon was recapitulated and analyzed. Based on the surface and sequence features, three candidate sites were selected:

FOLDON.wt:
(SEQ ID NO: 1)
GYIPEAPRDGQAYVRKDGEWVLLSTFL
FOLDON.S1:
(SEQ ID NO: 2)
GYIPEAPNDTQAYVRKDGEWVLLSTFL
FOLDON.S2:
(SEQ ID NO: 3)
GYIPEAPRDNQSYVRKDGEWVLLSTFL
FOLDON.S3:
(SEQ ID NO: 4)
GYIPEAPRDGQAYVRKDGNWTLLSTFL
Site 1:
RDG→NDT
Site 2:
GQA→NQS
Site 3:
EWV→NWT

### SEQUENCE LISTING

SEQ ID NO: 1
GYIPEAPRDGQAYVRKDGEWVLLSTFL
SEQ ID NO: 2
GYIPEAPNDTQAYVRKDGEWVLLSTFL
SEQ ID NO: 3
GYIPEAPRDNQSYVRKDGEWVLLSTFL
SEQ ID NO: 4
GYIPEAPRDGQAYVRKDGNWTLLSTFL

Mutant foldon domains having an amino acid sequence set forth in any one of SEQ ID NOs: 5, 6, and 7 will also be similarly evaluated. Further, studies will evaluate reduction in immune response to mutant foldon domains that each include two or more N-linked glycosylation sites as described here.

FOLDON.wt:
(SEQ ID NO: 1)
GYIPEAPRDGQAYVRKDGEWVLLSTFL
FOLDON.1:
(SEQ ID NO: 2)
GYIPEAPNDTQAYVRKDGEWVLLSTFL
(same sequence as FOLDON.S1)
FOLDON.2:
(SEQ ID NO: 5)
GYIPEAPRDGQAYVRNDTEWVLLSTFL
FOLDON.3:
(SEQ ID NO: 6)
GYIPEAPRDGQAYVRKNGTWVLLSTFL
FOLDON.4:
(SEQ ID NO: 4)
GYIPEAPRDGQAYVRKDGNWTLLSTFL
(same sequence as FOLDON.S3)
FOLDON.5:
(SEQ ID NO: 7)
GYIPEAPRDGQAYVRKDGEWVLLNTTL
Site 4:
KDG→NDT
Site 5:
DGE→NGT
Site 6:
STF→NTT

### SEQUENCE LISTING

SEQ ID NO: 5:
GYIPEAPRDGQAYVRNDTEWVLLSTFL
SEQ ID NO: 6:
GYIPEAPRDGQAYVRKNGTWVLLSTFL
SEQ ID NO: 7:
GYIPEAPRDGQAYVRKDGEWVLLNTTL

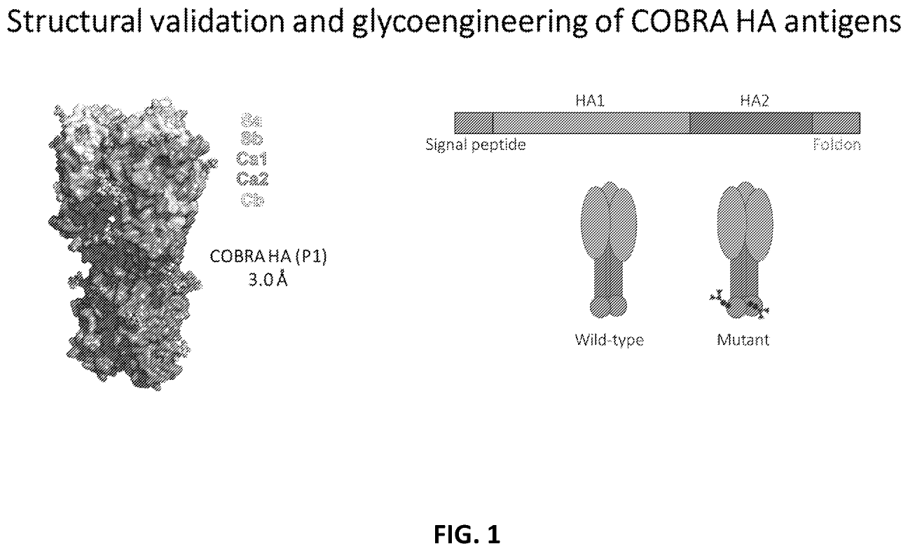

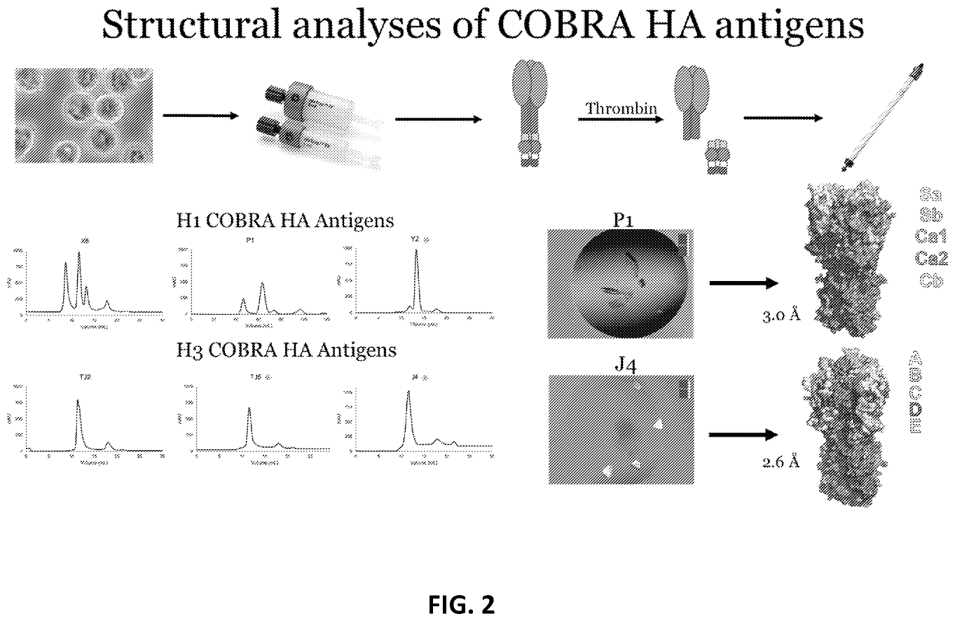

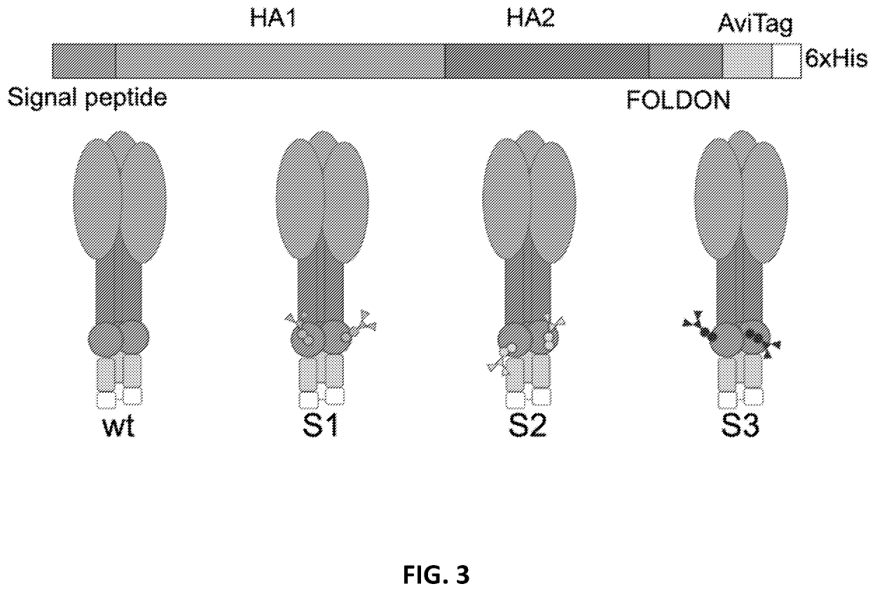

FIGS. 1-3 relate to structural validation and glycoengineering of COBRA HA antigens according to embodiments of the present disclosure. Schematically illustrated in FIG. 3, for example, is a polypeptide construct (top) comprising a signal peptide, an HA antigen comprising HA1 and HA2 domains, a foldon domain, a biotin tag (here, an AviTag), and a 6×His tag. Shown below in FIG. 3 are trimers of such polypeptides, where the foldon domain either has not been glycoengineered to include an N-linked glycosylation motif (wt) or where the foldon domain has been glycoengineered with the S1, S2 or S3 N-linked glycosylation motif as described herein followed by glycosylation of the motifs.

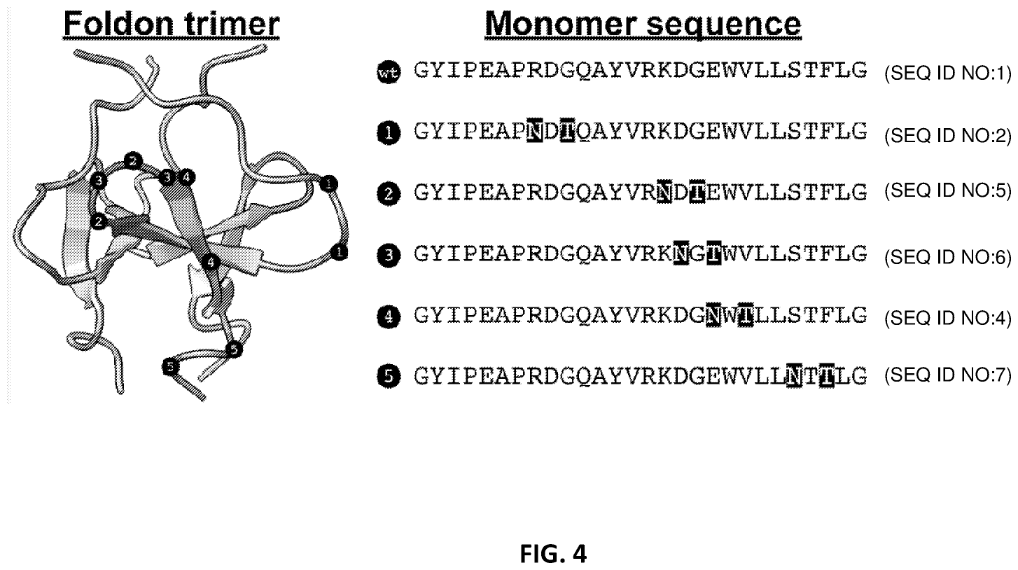

FIG. 4 provides a schematic for foldon glycoengineering. The figure graphics were generated using ChimeraX and Adobe Illustrator.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A polypeptide comprising a foldon domain comprising an N-linked glycosylation motif.

2. The polypeptide of claim 1, wherein the amino acid sequence of the foldon domain comprises 85% or greater identity to the amino acid sequence set forth in SEQ ID NO:1 (GYIPEAPRDGQAYVRKDGEWVLLSTFL), and
wherein the N-linked glycosylation motif is NXS/T, where X is any amino acid other than proline, and
optionally the N-linked glycosylation motif is NDT/S, NGT/S, NQT/S, NTT/S, or NWT/S.

3. The polypeptide of claim 2, wherein the N-linked glycosylation motif is NDT or NDS.

4. The polypeptide of claim 3, wherein the foldon domain comprises the amino acid sequence set forth in SEQ ID NO:2 (GYIPEAPNDTQAYVRKDGEWVLLSTFL).

5. The polypeptide of claim 2, wherein the N-linked glycosylation motif is NQS or NQT.

6. The polypeptide of claim 5, wherein the foldon domain comprises the amino acid sequence set forth in SEQ ID NO:3 (GYIPEAPRDNQSYVRKDGEWVLLSTFL).

7. The polypeptide of claim 2, wherein the N-linked glycosylation motif is NWT or NWS.

8. The polypeptide of claim 7, wherein the foldon domain comprises the amino acid sequence set forth in SEQ ID NO:4 (GYIPEAPRDGQAYVRKDGNWTLLSTFL).

9. The polypeptide of claim 1, wherein the foldon domain comprises the amino acid sequence set forth in:
SEQ ID NO: 5:
GYIPEAPRDGQAYVRNDTEWVLLSTFL;
SEQ ID NO: 6:
GYIPEAPRDGQAYVRKNGTWVLLSTFL;
or
SEQ ID NO: 7:
GYIPEAPRDGQAYVRKDGEWVLLNTTL.

10. The polypeptide of any one of claims 1 to 9, wherein the polypeptide consists essentially of the foldon domain.

11. The polypeptide of any one of claims 1 to 9, wherein the polypeptide consists of the foldon domain.

12. The polypeptide of any one of claims 1 to 9, wherein the foldon domain is fused to a heterologous amino acid sequence.

13. The polypeptide of claim 12, wherein the heterologous amino acid sequence is N-terminal to the foldon domain.

14. The polypeptide of claim 12 or claim 13, wherein the heterologous amino acid sequence is of an immunogenic polypeptide.

15. The polypeptide of claim 14, wherein the immunogenic polypeptide comprises a viral immunogenic polypeptide.

16. The polypeptide of claim 15, wherein the viral immunogenic polypeptide comprises an influenza hemagglutinin (HA) polypeptide, a human immunodeficiency virus (HIV) Env polypeptide, a respiratory syncytial virus (RSV) F polypeptide, or a SARS-CoV-2 spike polypeptide.

17. The polypeptide of claim 15, wherein the viral immunogenic polypeptide comprises an influenza HA polypeptide.

18. The polypeptide of claim 17, wherein the influenza HA polypeptide comprises an HA1 domain, an HA2 domain, or both.

19. The polypeptide of claim 17 or claim 18, wherein the influenza HA polypeptide comprises a Computationally Optimized Broadly Reactive Antigen (COBRA) influenza HA polypeptide.

20. The polypeptide of any one of claims 1 to 19, wherein the polypeptide comprises an affinity tag.

21. The polypeptide of any one of claims 1 to 20, wherein the polypeptide comprises a biotin tag.

22. The polypeptide of any one of claims 1 to 21, wherein the N-linked glycosylation motif is glycosylated.

23. The polypeptide of any one of claims 1 to 22, wherein the foldon domain comprises a plurality of N-linked glycosylation motifs.

24. The polypeptide of any one of claims 1 to 23, wherein the foldon domain comprises at least two N-linked glycosylation motifs selected from the group consisting of: NDT/S, NGT/S, NQT/S, NTT/S, and NWT/S.

25. The polypeptide of any one of claims 1 to 23, wherein the foldon domain comprises an amino acid sequence at least 85% identical to the amino acid sequence of SEQ ID NO:1 and comprises at least one of the following pairs of substitutions of the corresponding amino acids in SEQ ID NO: 1:
R8N and G10T/S;
G10N and A12T/S;
K16N and G18T/S;
D17N and E19T/S;
E19N and V21T/S; and
S24N and F26T.

26. The polypeptide of any one of claims 1 to 25, wherein the foldon domain comprises at least two of the following pairs of substitutions of the corresponding amino acids in SEQ ID NO:1:
R8N and G10T/S;
K16N and G18T/S; and
S24N and F26T;
or
G10N and A12T/S;
K16N and G18T/S;
E19N and V21T/S; and
S24N and F26T;
or
R8N and G10T/S;
G10N and A12T/S;
E19N and V21T/S; and
S24N and F26T.

27. A trimer of three polypeptides of any one of claims 1 to 26 trimerized via their foldon domains.

28. A trimer of claim 27, wherein the foldon domains have different sequences.

29. The trimer of claim 27 or 28, wherein one or more of the N-linked glycosylation motifs of the foldon domains are glycosylated.

30. An immunogenic composition comprising the trimer of any one of claims 27-29 and a pharmaceutically acceptable excipient.

31. A method of inducing an immune response in an individual, comprising administering to the individual an effective amount of the immunogenic composition of claim 30.

32. A nucleic acid encoding the polypeptide of any one of claims 1 to 26.

33. An expression vector comprising the nucleic acid of claim 32 operably linked to a promoter.

34. A recombinant host cell comprising the nucleic acid of claim 32 or the expression vector of claim 33.

35. A method of producing trimers comprising glycosylated foldon domains, the method comprising:
culturing a recombinant host cell comprising the expression vector of claim 33 under conditions suitable for expression of polypeptides comprising the foldon domains, glycosylation of the N-linked glycosylation motifs of the foldon domains, and trimerization of the polypeptides.

36. The method according to claim 35, further comprising purifying the trimers.

37. A kit comprising the immunogenic composition of claim 30, optionally wherein the composition is present in one or more (e.g., two or more) unit dosages.

38. The kit of claim 37, further comprising instructions for administering to an individual an effective amount of the immunogenic composition to induce an immune response in the individual.

39. A foldon domain comprising or consisting of an amino acid sequence having at least 85% identity to SEQ ID NO: 1 and comprising at least one of the following pairs of substitutions of the corresponding amino acids in SEQ ID NO: 1:
R8N and G10T/S;
G10N and A12T/S;
K16N and G18T/S;
D17N and E19T/S;
E19N and V21T/S; and
S24N and F26T.

40. The foldon domain of claim 39, wherein the foldon domain comprises at least two of the following pairs of substitutions of the corresponding amino acids in SEQ ID NO: 1:
R8N and G10T/S;
K16N and G18T/S; and
S24N and F26T;
or
G10N and A12T/S;
K16N and G18T/S;
E19N and V21T/S; and
S24N and F26T;
or
R8N and G10T/S;
G10N and A12T/S;
E19N and V21T/S; and
S24N and F26T.

41. The foldon domain of claim 39 or 40, wherein the foldon domain comprises or consists of the amino acid sequence set forth in SEQ ID NO: 2, 3, 4, 5, 6 or 7.

42. A nucleic acid comprising a nucleotide sequence encoding the foldon domain of any one of claims 39-41 or as set forth in any one of claims 1-26.

43. A vector comprising the nucleic acid sequence of claim 42.

44. The vector of claim 43, comprising one or more restriction sites upstream of the nucleotide sequence, wherein the one or more restriction sites are configured for in-frame fusion of a nucleic acid encoding an immunogenic polypeptide.

45. A host cell comprising the vector of claim 43 or 44.

46. The host cell of claim 45, wherein the vector is a cloning vector and the host cell is a bacterial cell.

47. The host cell of claim 45, wherein the vector is an expression vector and the host cells is a mammalian cell.

48. The host cell of claim 47, wherein the mammalian cell is a CHO cell line.


SEQUENCE LISTING (7 sequences)

SEQ ID NO: 1 - protein, 27 aa, synthetic construct
GYIPEAPRDGQAYVRKDGEWVLLSTFL

SEQ ID NO: 2 - protein, 27 aa, synthetic construct
GYIPEAPNDTQAYVRKDGEWVLLSTFL

SEQ ID NO: 3 - protein, 27 aa, synthetic construct
GYIPEAPRDNQSYVRKDGEWVLLSTFL

SEQ ID NO: 4 - protein, 27 aa, synthetic construct
GYIPEAPRDGQAYVRKDGNWTLLSTFL

SEQ ID NO: 5 - protein, 27 aa, synthetic construct
GYIPEAPRDGQAYVRNDTEWVLLSTFL

SEQ ID NO: 6 - protein, 27 aa, synthetic construct
GYIPEAPRDGQAYVRKNGTWVLLSTFL

SEQ ID NO: 7 - protein, 27 aa, synthetic construct
GYIPEAPRDGQAYVRKDGEWVLLNTTL